(19) United States
(12) Patent Application Publication
(10) Pub. No.: US 2015/0190412 A1
(43) Pub. Date: Jul. 9, 2015

(54) Pharmaceutical composition for the treatment of Lyme-borreliosis

(71) Applicant: Wolfgang ANDRES-FISCHER, Germering (DE)

(72) Inventor: Wolfgang ANDRES-FISCHER, Germering (DE)

(21) Appl. No.: 14/583,126

(22) Filed: Dec. 25, 2014

(30) Foreign Application Priority Data
Jan. 8, 2014 (DE) 10 2014 200 109.9

(51) Int. Cl. A61K 31/675; A61K 36/64; A61K 36/74; A61K 36/28; A61K 35/612; A61K 47/12; A61K 47/22; A61K 31/385; A61K 31/197; A61K 31/728; A61K 38/39; A61K 31/714; A61K 31/593; A61K 36/23; A61K 47/14; A61K 36/70

(52) U.S. Cl. CPC A61K 31/675; A61K 36/70; A61K 36/64; A61K 36/74; A61K 36/28; A61K 35/612; A61K 47/12; A61K 47/22; A61K 31/385; A61K 31/197; A61K 31/728; A61K 38/39; A61K 31/714; A61K 31/593; A61K 36/23; A61K 47/14

(57) ABSTRACT

The present invention provides for a composition to be applied in the treatment of borreliosis and a kit. The composition includes four groups of components with which group A) the components 1000-5000 mgs Cat's Claw, 1000-3000 mgs Krill Oil or Kalamarine Oil or combinations of it, 200-600 mgs Magnesium Malate, 3000-5000 mgs ester C, 100-300 mgs Alpha-Lipoic Acid, 25-75 mgs Pyridoxyl-5-Phosphate (vitamin B6), 6000-14000 IU Cholecalciferol (vitamin D3), 2000-4000 mgs He Shou Wu, 1200-2500 mgs Purple Clovers Tea, 10-50 mls parsley juice, the group B) 2000-4000 mgs Monolaurine, 400-800 mgs N-Acetylcysteine, 100-300 mgs Hyaluronic acid with BioCell collagens II, 1-5 drops Teasel Root, the group C) 10-70 drops Spilanthes Acmella and the group D) 500-1500 μg Cyancobalamin (vitamin B12). The composition and the kit is suitable to be applied in the treatment of borreliosis with which the components of the group A) are administered once daily, those of the group B) twice daily, those of the group C) three times daily and that of the group D) every 14 days.

Borrelias are a type of gram-negative and extremely slim bacteria with several wide meanders. They are named after the French bacteriologist Amédée Borrel (1867-1936) and belong to the family of the spirochetes. Their meanders or spirals are irregular, flexible and movable. Especially this flexibility distinguishes Borrelias from the spirillums.

Infectious diseases that are caused by bacteria from the group of the Borrelias are generally called borreliosis. The illnesses occur in human beings and in all other mammals as well as birds and can cause diverse clinical symptoms by affecting all body tissues. The pathogen types which are significant for human medicine are Borrelia burgdorferi sensu stricto, B. garinii, B. afzelii and B. spielmanii as pathogens of the Lyme-borreliosis (or also Lyme disease), as well as B. recurrentis and B. duttonii, that can cause the Recurrent Fever.

A transfer of the borreliosis pathogen usually takes place through ticks and lice that contracted the virus from red deer and small wild rodents, for example mice or hedgehogs. Among the numerous tick types, the sign-ticks ixodes ricinus (castor bean tick) in Europe and ixodes scapularsi (bear tick) as well as ixodes pacificus (Western black-legged tick) in North America and ixodes persulcatus (taiga tick) in Asia are the main vectors for infecting human beings there, since these types are euryphag, i.e. they are parasites on a multiplicity of different hosts. Depending on the region, up to 30%, partially also up to 50% of the ticks are infected with B. burgdorferi. Transfers through mosquitoes or horseflies were also described. (Guideline Neuroborreliosis of the Gelman Society for Neurology; in AWMF on-line, status 2008).

In the temperate zones, the Lyme-borreliosis is the most frequently transmitted infectious disease through arthropods. In Germany there are an estimated 60,000 new cases annually.

The clinical course of the Lyme-borreliosis can be divided into three stages. Between infection and clinical manifestation, days or even years can pass. In the first stage (local infection), a few days or weeks after the infection, a concentric reddening of the skin, the so-called wandering redness (Erythema migrans), builds on the entry point and advances to the surrounding area. This first stage can heal without any consequences or can take its course without any symptoms, so that the illness doesn't manifest until the second or third stage.

In the second stage (spreading of the pathogen), the skin, the central and peripheral nervous system, the hearts as well as the musculoskeletal system are primarily affected. However, the liver, the spleen, the lungs and the testicles can also be affected. In Europe, the Meningopolyneuritis Garin-Bujadoux-Bannwarth (neuro-borreliosis) is the most frequent clinical manifestation. Weeks or months after the tick bite, the disease pattern primarily shows radiating pain, which precedes the other symptoms in this stage, especially at night. Cranial nerve deficits, particularly Nervus facialis, are also described, as well as deficits in the form of rhythm disturbances, joint and muscle pains, as well as tiredness and a clear feeling of being ill, that are frequently also equated with flu symptoms.

In the third stage (late stage), a chronic disease pattern usually appears months or years after the infection and shows itself through the pathognomonic Acrodermatitis chronica atrophicans (ACA) and through rheumatic ailments. The chronic neuro-borreliosis distinguishes itself through a chronic meningitis or encephalomyelitis with lymphocytic pleocytosis in the liquor cerebrospinalis (cerebrospinal fluid) durating for more than six months. In the continuation of the illness, approximately half of the untreated patients develop arthritis (so-called Lyme-arthritis), typically in the large joints of the lower extremities. Shoulder and elbow joint inflammations are also known to occur, as well as the small finger joints being affected, especially in Europe.

Since a B. burdorferi infection does not guarantee immunity, reinfections can occur. In the course of the illness, specific antibodies as well as T-cells are built, whereupon the antibodies can be verified in laboratories, for example through indirect immune-fluorescence or Westemblot and ELISA. A two-step test is usually conducted, due to the high error rate in determining the infection. An ELISA-Test is conducted first, which can, however, show cross-reactions or polyclonal antibody stimulations through other pathogens and can therefore be falsely positive. The result is then verified and confirmed by an Immuno or Westemblot. If the ELISA is negative and there is a consistent clinical suspicion of borreliosis, conducting a Westemblot is recommended.

So far, treatment of borreliosis has been restricted to administering antibiotics for at least four weeks.

B. burgdorferi, for example is sensitive to β-Laktarn antibiotics and tetracyclines (Hahn, Falke, Kaufinami, Ullmann: “Medizinische Mikrobiologie and Infektiologie”; 4. Auflage, Springer Verlag). Due to the fact that especially in the early phase of an infection, no definite proof of illness except the wandering redness is possible, the occurrence of unspecific flu-like symptoms or joint pains after a tick bite pose the question of weighing the interests between the risks and side-effects of implementing a possibly superfluous antibiotic therapy lasting several weeks based on suspicion on the one hand, and on the other hand—if not implemented, or also in case of a possible failure of such a measure—the possible health, social and financial consequences of a long-tern chronic ailment, that could even lead to disability in extreme cases.

Therefore it is a task of the present invention to provide a new composition independent of antibiotics for the treatment of borreliosis.

### SUMMARY OF THE INVENTION

The present invention provides a composition for application in the treatment of borreliosis. The composition is divided into three groups of components. Group A) consists of 1000-5000 mgs cat's claw, 1000-3000 mgs Krill oil or Kalamarine oil or combinations of it, 200-600 mgs magnesium malate, 3000-5000 mgs ester C, 100-300 mgs alpha-lipoic acid, 25-75 mgs pyridoxyl-5-phosphate (vitamin B6), 6000-14000 IU cholecalciferol (vitamin D3), 2000-4000 mgs He Shou Wu, 1200-2500 mgs red clover tea and 10-50 mls parsley juice. Group B) consists of 2000-4000 mgs monolaurine, 400-800 mgs N-Acetylcysteine, 100-300 mgs Hyaluronicic acid with BioCell collagen II and 1-5 drops teasel root. Group C) consists of 10-70 drops Spilanthes Acmella and in group D) 500-1500 μgs cyanocobalamin (vitamin B12). The components of group A) administered once a day, the components of group B) administered twice a day, the component of group C) administered three times a day and the component of group D) once in 14 days.

In a preferential implementation form, the borreliosis is a Lyme-borreliosis.

Monolaurine, N-Acetylcysteine, hyaluronic acid with BioCell collagen II and teasel root (group B) are administered preferably in the morning and in the evening. Especially preferred is that between 15 and 6 hours lie between the two dispensations, more preferentially between 13 and 8 hours, and the most preferred between is 12 and 10 hours.

Spilanthes Acmella (group C) is administered preferably in the morning, at noon and in the evening. Especially preferred is that between 14 and 2 hours lie between the three dispensations, more preferentially between 11 and 4 hours, and the most preferred is between 8 and 6 hours.

Cyanocobalamin (group D) is dispensed preferably on the first, second, third, fourth, fifth, sixth, seventh, eighth, ninth, tenth, eleventh, twelfth, thirteenth or fourteenth day after the first dispensation of the components of groups A), B) and C).

The composition is administered preferably orally. Alternatively, the composition can also be administered intravenously. All materials can be administered simultaneously or successively. Cyanocobalamin is administered preferably by injection, preferably through subcutaneous, intramuscular or intravenous injection.

In a preferred implementation form, the composition in group A) consists of 1500-4500 mgs cat's claw, 1500-2500 mgs Krill oil or Kalamarine oil or combinations of it, 300-500 mgs magnesium malate, 3500-4500 mgs ester C, 150-250 mgs alpha-lipoic acid, 40-60 mgs pyridoxyl-5-phosphate (vitamin B6), 8000-12000 IU cholecalciferol (vitamin D3), 2500-3500 mgs He Shou Wu, 1500-2250 mgs red clover tea, 20-40 mls parsley juice, in group B) 2500-3500 mgs monolaurine, 500-700 mgs N-Acetylcysteine, 150-250 mgs hyaluronic acid with BioCell collagen II and 1-4 drops teasel root, in group C) 20-60 drops Spilanthes Acmella and in group D) 800-1200 mgs cyanocobalamin (vitamin B12).

Especially preferably, the composition in group A) consists of 2000-4000 mgs cat's claw, 2000 mgs Krill oil or alternatively Kalamarine oil, 400 mgs magnesium malate, 4000 mgs ester C, 200 mgs alpha lipoic acid, 50 mgs pyridoxyl-5-phosphate, 10.000 IU cholecalciferol, 3000 mgs He Shou Wu, 1500-2250 mgs red clover tea, 30 mls parsley juice, in group B, 3000 mgs monolaurine, 600 mgs N-Acetylcysteine, alternatively 200 mgs Hyaluronic acid with BioCell collagen II and 2-3 drops teasel root, in group C) 40 drops Spilanthes Acmella and in group D) 1000 μg cyanocobalamin (vitamin B12).

The present invention also provides a kit that includes the following groups of components: Group A) with 1000-5000 mgs cat's claw, 1000-3000 mgs Krill oil or Kalamarine oil or combinations of it, 200-600 mgs magnesium malate, 3000-5000 mgs ester C, 100-300 mgs alpha-lipoic acid, 25-75 mgs pyridoxyl-5-phosphate (vitamin B6), 6000-14000 IU cholecalciferol (vitamin D3), 2000-4000 mgs He Shou Wu, 1200-2500 mgs red clover tea, 10-50 mls parsley juice, group B) with 2000-4000 mgs monolaurine, 400-800 mgs N-Acetylcysteine, 100-300 mgs Hyaluronic acid with BioCell collagen II and 1-5 drops teasel root, the group C) 10-70 drops Spilanthes Acmella and in group D) 500-1500 μg cyanocobalamin (vitamin B 12), as well as instructions for the application and dispensation of the individual groups A)-D).

In a preferred implementation form, the kit includes the following groups of components: Group A) 1500-4500 mgs cat's claw, 1500-2500 mgs Krill oil or Kalamarine oil or combinations of it, 300-500 mgs magnesium malate, 3500-4500 mgs ester C, 150-250 mgs alpha-lipoic acid, 40-60 mgs pyridoxyl-5-phosphate (vitamin B6), 8000-12000 IU cholecalciferol (vitamin D3), 2500-3500 mgs He Shou Wu, 1500-2250 mgs red clover tea, 20-40 mls parsley juice, group B) 2500-3500 mgs monolaurine, 500-700 mgs N-Acetylcysteine, 150-250 mgs Hyaluronic acid with BioCell collagen II and 1-4 drops teasel root, the group C) 20-60 drops Spilanthes Acmella and in group D) 800-1200 μg cyanocobalamin (vitamin B12).

In an especially preferred implementation form, the kit includes the following groups of components: Group A) 2000-4000 mgs cat's claw, 2000 mgs Krill oil, alternatively Kalamarine oil, 400 mgs magnesium malate, 4000 mgs ester C, 200 mgs alpha lipoic acid, 50 mgs pyridoxyl-5-phosphate, 10.000 IU cholecalciferol, 3000 mgs He Shou Wu, 1500-2250 mgs red clover tea, 30 mls parsley juice, group B) 3000 mgs monolaurine, 600 mgs N-Acetylcysteine, 200 mgs Hyaluronic acid with BioCell collagen II and 2-3 drops teasel root, group C) 40 drops Spilanthes Acmella and in group D) 1000 pg cyanocobalamin (vitamin B 12).

The instructions in the kit, in accordance with the invention, can include instructions for the treatment of borreliosis and can specify that the components of group A) should be administered once a day, those of group B) twice a day, those of group C) three times a day and those of group D) every 14 days.

The kit, in accordance with the invention, is suitable for the treatment of borreliosis, preferably the Lyme-borreliosis.

In the kit, in accordance with the invention, the instructions can specify that the components of group B) should be administered in the morning and in the evening.

In the kit, in accordance with the invention, the instructions can specify that the component of group C) should be administered in the morning, at noon and in the evening.

In the kit, in accordance with the invention, the instructions can specify that the component of group D) should be administered on the first, second, third, fourth, fifth, sixth, seventh, eighth, ninth, tenth, eleventh, twelfth, thirteenth or fourteenth day after the primary dispensation of the components of groups A), B) and C).

### DETAILED DESCRIPTION OF THE INVENTION

The present invention provides a composition that has proven itself to be extremely effective in the treatment of borreliosis, preferentially Lyme-borreliosis. The individual components of the composition can be divided into four groups, A) to D) structures, where group A) contains cat's claw, Krill oil or Kalamarine oil or combinations of it, magnesium malate, ester C, alpha-lipoic acid, pyridoxyl-5-phosphate (vitamin B6), cholecalciferol (vitamin D3), He Shou Wu, red clover tea and parsley juice, group B) contains monolaurine, N-Acetylcysteine, Hyaluronic acid with BioCell collagen II and teasel root, group C) contains Spilanthes Acmella and group D) contains cyanocobalamin (vitamin B12).

The components of group A) are administered once a day, the components of group B) are administered twice a day, the component of group C) is administered three times a day and the component of group D) once within 14 days.

The quantity of cat's claw in the composition amounts to 1000-5000 mgs, preferably 1500-4500 mgs, more preferably 1750-4250 mgs, even more preferably 1900-4100 mgs and most preferably 2000-4000 mgs. Cat's claw, (scientific name Uncaria tomentosa), can be obtained from Natural Oranics, Inc., 548 Broadhollow Road, Melville, N.Y. 11747, USA. Cat's claw is a plant extract in capsule form, which is won from the inner bark.

The quantity of Krill oil or Kalamarine oil or combinations of it in the composition amounts to 1000-3000 mgs, preferably 1250-2750 mgs, more preferably 1500-2500 mgs, even more preferably 1750-2250 mgs and most preferably 2000 mgs. Krill oil as well as Kalamarine oil can be obtained from Greenvitamins N.V., Kruisdonk 66, NL-6222 PH Maastricht, Netherlands.

The quantity of magnesium malate in the composition amounts to 200-600 mgs, preferably 300-500 mgs, more preferably 350-450 mgs, even more preferably 375-425 mgs and most preferably 400 mgs.

The quantity of ester C in the composition amounts to 3000-5000 mgs, preferably 3500-4500 mgs, more preferably 3750-4250 mgs, even more preferably 3900-4100 mgs and most preferably 4000 mgs. Ester C is available in tablet form from Solgar Inc., 500 Willow Tree Road, Leonia, N.J. 07605, USA, A 500 mgs tablet contains 500 mgs vitamin C as ester C-calcium Ascorbate, 60 mgs calcium as calcium Ascorbate, 25 mgs citrus Bioflavonoid complex, 10 mgs acerola, 10 mgs fruits of rose hip and 5 mgs rutin. (These statements refer to statements from Solar Inc.)

The quantity of alpha-lipoic acid in the composition amounts to 100-300 mgs, preferably 150-250 mgs, more preferably 170-220 mgs, even more preferably 190-210 mgs and most preferably 200 mgs.

The quantity of pyridoxyl-5-phosphate (vitamin B6) in the composition amounts to 25-75 mgs, preferably 30-70 mgs, more preferably 40-60 mgs, even more preferably 45-55 mgs and most preferably 50 mgs.

The quantity of cholecalciferol (vitamin D3) in the composition amounts to 6000-14000 IU, preferably 8000-12000 IU, more preferably 9000-11000 IU, even more preferably 9500-10500 IU and most preferably 10000 IU.

The quantity of He Shou Wu in the composition amounts to 2000-4000 mgs, preferably 2500-3500 mgs, more preferably 2750-3250 mgs, even more preferably 2900-3100 mgs and most preferably 3000 mgs. He Shou Wu, also known as Fo—Ti in the traditional Chinese medicine, is won from the root of the Polygonum multiflorum and can be obtained in capsulated form, for example, from BodyMe Ltd, Unit 12 Rockhill Estate, Wellsway, Keynsham, Bristol, BS31 1PF.

The quantity of red clover tea in the composition amounts to 1200-2500 mgs, preferably 1500-2250 mgs, more preferably 1750-2000 mgs, even more preferably 1800-1900 mgs and most preferably 1850 mgs. Red clover tea consists of red clover blossoms and coarse-cut and can be obtained, for example, from AURICA Naturheilmittel and Naturwaren GmbH, Piittlinger St. 121, 66773 Schwalbach, Germany.

The quantity of parsley juice in the composition amounts to 10-50 mls, preferably 20-40 mls, more preferably 25-35 mls, even more preferably 27-32 mls and most preferably 30 mls. Parsley juice can be obtained in the form of a pressed parsley herb juice from the Walther Schoenenberger Pflanzensaftwerk GmbH & Co. KG, Hutwiesenstraβe 14, 71106 Magstadt, Germany.

The quantity of monolaurine in the composition amounts to 2000-4000 mgs, preferably 2500-3500 mgs, more preferably 2750-3250 mgs, even more preferably 2900-3100 mgs and most preferably 3000 mgs. Monolaurine can be obtained from Aviva Natural Health Solutions, 1224 St. James Street, Winnipeg, MB Canada R3H OL1.

The quantity of N-Acetylcysteine in the composition amounts to 400-800 mgs, preferably 500-700 mgs, more preferably 550-650 mgs, even more preferably 575-625 mgs and most preferably 600 mgs.

The quantity of Hyaluronic acid with BioCell collagen 11 in the composition amounts to 100-300 mgs, preferably 150-250 mgs, more preferably 175-225 mgs, even more preferably 180-220 mgs and most preferably 200 mgs. Hyaluronic acid with BioCell collagen II can be obtained from Vitabay B.V., Avenue Ceramique 221, Maastricht, NL-6221 KX, Netherlands.

The quantity of teasel root in the composition amounts to 1-7 drops, preferably 1-6 drops, more preferably 1-5 drops, even more preferably 1-4 drops and most preferably 2-3 drops. Teasel root can he obtained, for example, from Nuhrovia GmbH, Birkenweg 9, A-5145 Neukirchen, Austria.

The quantity of Spilanthes Acmella in the composition amounts to 10-70 drops, preferably 20-60 drops, more preferably 30-50 drops, even more preferably 35-45 drops and most preferably 40 drops. Spilanthes Acmella can be obtained from Herb Phann, 20260 Williams Hwy., Williams, Oreg. 97544, USA.

This is a plant extract from the newly blossoming plant herb or the plant root.

The quantity of cyanocobalamin (vitamin B12) in the composition amounts to 500-1500 μgs, preferably 600-1400 μgs, more preferably 800-1200 μgs, even more preferably 900-1100 μgs and most preferably 1000 μgs.

Magnesium malate, alpha-lipoic acid, vitamins B6, B12, D3 as well as N-Acetylcysteine are available in Germany mainly in drugstores or phaimacies.

The present invention is especially suitable for prophylactic antibiotic therapy when borreliosis is suspected, since no side-effects have become known in connection with the individual components or their combination until now.

The kit, in accordance with the invention, includes all components of groups A) to D), as well as instructions for administering the groups A) to D) as presented above. Therefore, the kit is especially suitable for the treatment of borreliosis, preferably of Lyme-borreliosis. The quantities and statements of origin of the components of the individual groups A) to D) of the composition described above are also valid for the kit in accordance with the invention. In addition, the displayed dispensation forms are also applicable to the kit.

### EXAMPLES

The composition appropriate to the invention was administered to 5 persons (1 male, 4 female) in the age of 37 to 73 years. The average age of the patients was 58 years.

The therapy corresponded to the stated composition and existed hereinafter from: oral 1 times daily 2000-4000 mgs cat's claw, 2000 mgs krill oil, 400 mgs magnesium malate, 4000 mgs ester C, 200 mgs alpha lipoic acid, 50 mgs pyridoxyl-5-phosphate, 10.000 IU vitamine D3, 3000 mgs He Shou Wu, 1500-2250 mgs purple clover tea, 30 mls parsley juice 2× daily 3000 mgs Monolaurine, 600 mgs N-Acetylcysteine, 200 mgs hyaluronic acid with BioCell collagens II and 2-3 drops of teasel root; 3×daily 40 drops of Spilanthes Acmella and every 14 days 1000 μgs cyancobalamin intramuscular or subcutan. Prerequisite was a strict observance of the pre-determined administration of the drugs.

The patient's exit-symptoms showed a wide spectrum since the pathogens gather in the spinal-liquid, joint-liquid, eye-liquid are hiding there as well as diffusing in the blood circulation all over the body.

The diagnose was made by taking the patient's history and the clinical symptoms on the basis of blood-tests and/or biopsies with serology (by the means of specific antibodies with immunoassays ELISA or CLIA, specific immunoblot with corresponding Immunoglobine-and banding pattern, liquor—and/or joint-puncture with PCR, propagation of the pathogen Borrelia burgdorferi or assay of the chemokines CXCL13. In all patients could be proven the pathogen Borrelia burgdorferi.

The duration of therapy-application amounted in the cut to 5 months. A clear improvement in reference to the symptoms was described by all the patients at the end of the therapy. The pathogens were no longer detectable in the following blood-tests.

Patient 1: 37 year old male. Therapy duration: 3½ months. The patient had the following symptoms at the beginning of therapy: Tiredness and nausea, alternating headaches, sore throats, short cold, no fever episodes. After termination of the therapy the patient was free of complaints.

Patient 2: 44 year old female. Therapy duration: 4 months. The patient had the following symptoms at the beginning of therapy: Inner restlessness, nervousness, insomnia, headache, stomach aches. After termination of the therapy the patient was free of complaints.

Patient 3: 71 year old female. Therapy duration: 5 months. The patient had the following symptoms at the beginning of therapy: Alternating joint pain (elbows, wrist, ankle, knee), diarrhea, nausea, digestive problems, stomachaches, headaches, tiredness, insomnia, inner restlessness, fever. After termination of the therapy the patient was free of complaints.

Patient 4: 65 year old female. Therapy duration: 5½ months. The patient had the following symptoms at the beginning of therapy: Alternating joint-pains, erythematic skin appearances, occasional headaches, inner restlessness, nausea and low elevated body temperature. After termination of the therapy the patient was free of complaints.

Patient 5: 73 year old female. Therapy duration: 5 months. The patient had the following symptoms at the beginning of therapy: Alternating joint-pains, joint-deformities, mobility difficulties, heart valve failure, neuropathy, physical weakness, recurrent fever, concentration deficits. After termination of the therapy the patient was free of complaints.

1. Composition containing the following components:
Group A) with
1000-5000 mgs cat's claw
1000-3000 mgs Krill oil or Calamarine Oil or combinations of it
200-600 mgs magnesium malate
3000-5000 mgs ester C
100-300 mgs alpha-lipoic acid
25-75 mgs pyridoxyl-5-phosphate (vitamin B6)
6000-14000 IU vitamin D3 (vitamin D3)
2000-4000 mgs He Shou Wu
1200-2500 mgs purple clover tea
10-50 mls parsley juice
Group B) with
2000-4000 mgs monolaurine
400-800 mgs N-Acetylcysteine
100-300 mgs hyaluronic acid with. BioCell collagens II
1-5 drops teasel root
Group C) with
10-70 drops Spilanthes Acmella and
Group D) with
500-1500 μgs Cyancobalamin (vitamin B12)
to use in the treatment of borreliosis, the components of group A) being administered once daily, Group B) components twice daily, the group C) components three times daily and those of the group D) every 14 days.

2. Composition for use according to claim 1, wherein Borreliosis corresponds to Lyme-borreliosis.

3. Composition for use according to claim 1, wherein components of the group B) are administered in the morning and in the evening.

4. Composition according to claim 1, wherein components of the group C) are administered in the morning, at noon and in the evening.

5. Composition for use according to claim 1, with the component of the group D) being administered at the first, second, third, fourth, fifth, sixth, seventh, ninth, tenth, eleventh, twelfth, thirteenth or fourteenth day after the primary administration of the components of the groups A), B), and C).

6. Composition for use according to claim 1, the composition being administered orally.

7. Composition for use according to claim 1, the component of the group D) being administered by subcutaneous, intramuscular or intravenous injection.

8. Composition for use according to claim 1, containing the following groups:
Group A) with
1500-4500 mgs cat's claw
1500-2500 mgs Krill oil or Calamarine Oil or combinations of it
300-500 mgs magnesium malate
3500-4500 mgs ester C
150-250 mgs alpha-lipoic acid
40-60 mgs pyridoxyl-5-phosphate (Vitamin B6)
8000-12000 IU Vitamin D3 (Vitamin D3)
2500-3500 mgs He Shou Wu
1200-2250 rugs purple clover tea
20-40 mls parsley juice
Group B) with
2500-4000 mgs monolaurine
500-700 mgs N-Acetylcysteine
150-250 mgs hyaluronic acid with BioCell collagens II
1-4 drops teasel root
Group C) with
20-60 drops Spilanthes Acmella, and
Group D) with 800-1200 μg extrinsic factors (vitamin B12).

9. Composition to use according to claim 8, including
Group A) with
2000-4000 mgs cat's claw
2000 mgs Krill Oil or Kalamarine Oil or combinations of it
400 mgs Magnesium malate
4000 mgs ester C
200 mgs Alpha-Lipoic Acid
50 mgs Pyridoxyl-5-Phosphate (Vitamin B6)
10000 IU Cholecalciferol (Vitamin D3)
3000 mgs He Shou Wu
1500-2250 mgs Purple Clover Tea
30 mls parsley juice
Group B) with
3000 mgs Monolaurine
600 mgs N-Acetylcysteine
200 mgs hyaluronic acid with BioCell collagens II
2-3 drops Teasel Root
Group C) with
40 drops Spilanthes Acmella and
Group D) with
1000 μg Cyanocobalamin (Vitamin B12).

10. Kit containing the following groups of components:
Group A) with
1000-5000 mgs Cat's Claw
1000-3000 mgs Krill Oil or Kalamarine Oil or combinations of it
200-600 mgs Magnesium malate
3000-5000 mgs ester C
100-300 mgs Alpha-Lipoic Acid
25-75 mgs Pyridoxyl-5-Phosphate (Vitamin B6)
6000-14000 IU Cholecalciferol (Vitamin D3)
2000-4000 mgs He Shou Wu
1200-2500 mgs Purple Clovers Tea
10-50 mls parsley juice
Group B) with
2000-4000 mgs Monolaurine
400-800 mgs N-Acetylcysteine
100-300 mgs Hyaluronic acid with BioCell collagens II
1-5 drops Teasel Root
Group C) with
10-70 drops Spilanthes Acmella, and
Group D) with
500-1500 μg Cyancobalamin (Vitamin B12),
according to the instruction to apply in the individual groups A)-D.

11. Kit according to claim 10, containing the following groups of components:
Group A) with
1500-4500 mgs Cat's Claw
1500-2500 mgs Krill Oil or Kalamarine Oil or combinations of it
300-500 mgs Magnesiums Malate
3500-4500 mgs ester C
150-250 mgs Alpha-Lipoic Acid
40-60 mgs Pyridoxyl-5-Phosphate (vitamin B6)
8000-12000 IU Cholecalciferol (vitamin D3)
2500-3500 mgs He Shou Wu
1500-2250 mgs Purple Clovers Tea
20-40 mls parsley juice
Group B) with
2500-3500 mgs Monolaurine
500-700 mgs N-Acetylcysteine
150-250 mgs Hyaluronic Acid with BioCell collagens II and
1-4 drops Teasel Root
Group C) with
20-60 drops Spilanthes Acmella, and
Group D) with
800-1200 μg Cyancobalamin (vitamin B12).

12. Kit according to claim 11, containing the following group components :
Group A) with
2000-4000 mgs Cat's Claw
2000 mgs Krill Oil or Kalamarine Oil or combinations of it
400 mgs Magnesium Malate
4000 mgs ester C
200 mgs Alpha-Lipoic Acid
50 mgs Pyridoxyl-5-Phosphate (Vitamin B6)
10000 IU Cholecalciferol (Vitamin D3)
3000 mgs He Shou Wu
1500-2250 mgs Purple Clovers Tea
30 mls parsley juice
Group B) with
3000 mgs Monolaurine
600 mgs N-Acetylcysteine
200 mgs Hyaluronic Acid with BioCell collagens II
2-3 drops Teasel Root
Group C) with
40 drops Spilanthes Acmella and
Group D) with
1000 μg Cyancobalamin (Vitamin B12).

13. Kit according to one of the claim 10, containing a treatment instruction for borreliosis is contained, said instruction including the components of the group A) are administered once daily, those of the group B) twice daily, those of the group C) three times daily and those of the group D) every 14 days.

14. Kit according to claim 13, wherein Borreliosis corresponds to Lyme-borreliosis.

15. Kit according to claim 13, with components of the group B) being administered in the morning and in the evening.

16. Kit according to claim 13, with components of the group C) being administered in the morning, at noon and in the evening.

17. Kit according to claim 13, with the component of the group D) administered at the first, second, third, fourth, fifth, sixth, seventh, ninth, tenth, eleventh, twelfth, thirteenth or fourteenth day after the primary dispensation of the components of the groups A), B), and C).
